(19) United States
(12) Patent Application Publication
(10) Pub. No.: US 2021/0361372 A1
(43) Pub. Date: Nov. 25, 2021

(54) SPECIMEN ZONE MAT

(71) Applicant: The Penn State Research Foundation, University Park, PA (US)

(72) Inventors: Jennifer E. Butch, Hershey, PA (US); Kerri A. Wolverton, Hershey, PA (US)

(21) Appl. No.: 17/059,636

(22) PCT Filed: May 31, 2019
(86) PCT No.: PCT/US2019/034835

§ 371 Date: Nov. 30, 2020

Related U.S. Application Data
(60) Provisional application No. 62/679,258, filed on Jun. 1, 2018.

(51) Int. Cl. A61B 50/20; A61B 50/30; B01L 9/00

(52) U.S. Cl. CPC A61B 50/20; A61B 50/30; B01L 2300/165; B01L 2300/021; B01L 2300/123; B01L 9/00

(57) ABSTRACT

A specimen zone mat that sets a precedent and standard to improve specimen handling and recognition includes: a body portion that is flexible and pliable, and having a first surface made of a writable material such that a user is able to write directly thereon and a second surface opposite to the first surface and made of a non-slip material, in which the body portion is sterile.

### CROSS-REFERENCE TO RELATED APPLICATIONS

This application claims priority to U.S. provisional patent application No. 62/679,258, filed on Jun. 1, 2018, incorporated herein by reference in its entirety.

### TECHNICAL FIELD

The present disclosure relates to a specimen zone mat for use in organizing one or more specimens in one or more designated areas, Such mats can be used in surgical rooms when specimens are taken from a subject, for example.

### BACKGROUND

In a surgical environment, e.g., an operating room (OR) or the like, specimens may be collected from a subject, e.g., a human patient, and placed and organized on a surgical table, Traditionally, surgeons, nurses, physicians or other workers are trained to draw grids directly onto a paper back table, if the room allows. However, due to a cluttered and limited space, people may not recognize that specimens are present. There is also a difficulty in tracking multiple specimen designation sites. Further, paper drapes and clarity of writing may become compromised, which can lead to confusion in differentiating the specimens. In addition, there is no set mandatory practice or standards on how to arrange or designate locations on the back table for the collected specimens.

Therefore, there is a need for a designated area for placing specimens from a subject in an organized way for members in a surgical room.

### SUMMARY OF THE DISCLOSURE

An aspect of the present disclosure includes a specimen zone mat for use in organizing one or more specimens in a designated area. Such mats can improve specimen handling, recognition and identification.

According to an aspect of the present disclosure, a specimen zone mat can include: a body portion that is flexible and pliable, and having a first surface made of a writable material such that a user is able to write directly thereon and a second surface opposite to the first surface in which the second surface is a non-slip surface.

Advantageously, at least the first surface which contacts the specimen, and preferably the entire specimen mat, is sterile. Further, the specimen zone mats of the present disclosure can include designated zone areas, such as a grid or boxes or circles or ovals, and with or without labels or with or without areas for labels.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. Accordingly, the drawings and description are to be regarded as illustrative in nature, and not as restrictive.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a schematic top view illustrating a specimen zone mat according to an embodiment of the present disclosure.

### DETAILED DESCRIPTION OF THE DISCLOSURE

Hereinafter, an exemplary embodiment in the present disclosure will be described with reference to the accompanying drawings. In the accompanying drawings, shapes, sizes, and the like, of components may be exaggerated or shortened for clarity.

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

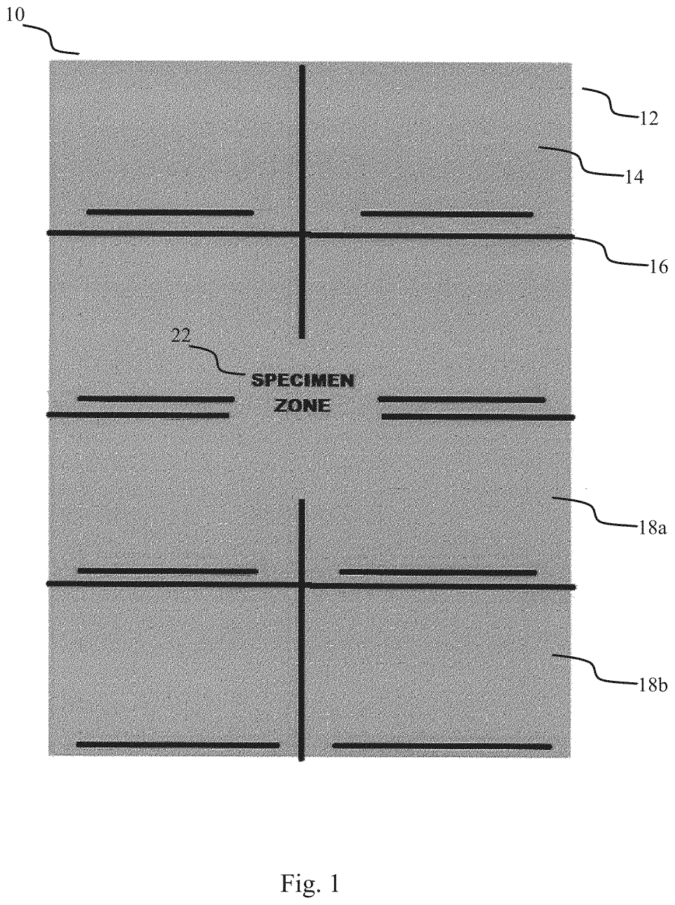

FIG. 1 is a schematic top view illustrating a specimen zone mat according to an embodiment of the present disclosure. As shown in the figure, a specimen zone mat 10 can include a body portion 12 that is flexible and pliable. The body portion 12 of the specimen zone mat 10 includes a first surface 14 that is made of a writable material, such that a user can write directly on the surface with, for example, permanent markers without smearing. Such writeable materials can comprise, for example, a polyolefin, e.g., polyethylene, polypropylene, etc., or a polyester, for example a clear polyester, or a polyvinyl chloride based material or a rubber such as polybutylene, polyisoprene, etc., or the like. The body portion can be a single material or a combination of materials including a laminated material and the body portion can be made of the same material as the second surface. A laminating material may comprise for example a construction adhesive.

The first surface 14 of the specimen zone mat 10 can have one or more grid lines 16 that provide a plurality of designated sections (18a, 18b, etc.) that can be used to organize or arrange one or more specimens at a specific location on the mat. Preferably, the one or more grid lines 16 may divide the first surface 14 into six to eight designated sections for placing one or more specimens thereon. Although the embodiment of FIG. 1 illustrates a grid to divide the mat, specimen zone mats of the present disclosure can be divided, with or without grid lines, into designated zone areas, such as boxes or circles or ovals with or without labels or with or without lines for labels. During a surgical procedure or procedure in which specimens are taken from a subject such as from a human subject, a specimen zone mat can greatly facilitate organization and reduce errors in accounting for and identifying specimens.

The body portion 12 may further include a second surface 20 (not shown for illustrative convenience) which is opposite to the first surface 14. Preferably, the second surface is a non-slip surface. Such a surface can be fabricated by texturing the surface and/or can be made of a non-slip material, such as a rubber material, e.g., polybutylene, polyisoprene or a polyolefin, e.g., polyethylene, polypropylene, etc., or a polyvinyl chloride based material, or the like. Such a surface can also include or alternatively have an adhesive material to provide non-slippage and/or capabilities to affix the mat to a surgical table or any surface designated within the sterile field for a specimen. By using such materials, specimen zone mats of the present disclosure do not readily move once in place.

As another example, the first and second surfaces 14 and 20 may be made of a mixture of both writable and non-slip materials.

The specimen zone mat 10 may be sterilized and individually packed for one time use with an ability to easily dispense onto a surgical field. Sterilization parameters may be varied by the manufacturers and may not be limited. In an aspect of the present disclosure, the specimen zone mat is in a sterile, single use package. The specimen zone mat can itself also be sterile in the sterile, single use package.

The specimen zone mat is preferably in the shape of a sheet and can be dimensioned as such. Being in the shape of a sheet allows the specimen mat to lay flat on a table, such as a table in a surgical room. Further for easy handling by a member of a surgical team, the specimen mat can be dimensioned such that the body portion has a size of no more than about 1 meter (about 39.4 inches) in length and no more than about 1 meter (about 39.4 inches) in width and no more than about 10 cm (about 4 inches) in thickness. In an aspect of the present disclosure, the specimen mat can be dimensioned such that the body portion has a size of no more than about 8 cm to about 14 cm in length and no more than about 8 cm. to about 14 cm in width and no more than about 8 cm, 6 cm, 4 cm and 2 cm and even less than about 1 cm in thickness. For example, the body portion 12 of the specimen zone mat 10 can have an approximate size of 30.5 cm (about 12 inches) in length and about 25.5 cm (about 10 inches) in width and about 0.635 cm (0.25 inches) in thickness. For easy storage, the specimen mat can be foldable upon itself to reduce its size and for packaging in a single use package which is preferably sterilized. In addition, the specimen zone mat of the present disclosure can have any one or more features of moisture resistant, impermeable to fluids such as water based mixtures typical of specimens, e.g., blood or body fluids, non-degradable such that the mat does not contaminate specimens, non-breakable, etc.

Another advantage of the specimen mat of the present disclosure is that it can include visual features that can signal cues to the members of a surgical team or relief staff. For example, specimen zone mats of the present disclosure be colored such as light purple or green and/or in bright colors and even in a neon color such as lime green, so that it can serve as a visual cue for users in the surgical room to both identify the mat and wherein a specimen on the mat can be readily observed. In addition, more than one color can be used as well as shading to enhance contrast, brightness and/or saturation. The specimen zone mats of the present disclosure can be label for identification for its purpose. Further, the specimen zone mats of the present disclosure can include one or more zone areas designating locations for various specimens to be placed.

In an aspect of the present disclosure and with reference to FIG. 1, the first surface 14 can include a label to readily identify the mat to the users in the surgical room. As an example, the specimen zone mat can include a label of “Specimen Zone” (element 22) in a center portion of the first surface 14 in the embodiment illustrated in FIG. 1.

As set forth above, a specimen zone mat according to the exemplary embodiment in the present disclosure can provide an efficient way to organize and identify multiple specimens in a surgical room or any other similar environment. This specimen zone mat may be easily recognized by any member in the surgical room. This specimen zone mat can be further used at any location convenient to an operating room for a procedure on a subject, e.g., a human subject or other subject in which one or more specimens are taken.

Only the preferred embodiment of the present invention and examples of its versatility are shown and described in the present disclosure. It is to be understood that the present invention is capable of use in various other combinations and environments and is capable of changes or modifications within the scope of the inventive concept as expressed herein. Thus, for example, those skilled in the art will recognize, or be able to ascertain, using no more than routine experimentation, numerous equivalents to the specific substances, procedures and arrangements described herein, Such equivalents are considered to be within the scope of this invention, and are covered by the following claims.

What is claimed is:

1. A specimen zone mat for use in organizing one or more specimens in a designated area, comprising:
a body portion that is flexible and pliable, and having:
a first surface made of a writable material such that a user is able to write directly thereon; and
a second surface opposite to the first surface and being a non-slip surface, wherein the body portion is sterile.

2. The specimen zone mat of claim 1, wherein the first surface has one or more grid lines thereon.

3. The specimen zone mat of claim 2, wherein the one or re grid lines divide the first surface into a plurality of designated sections for placing one or more specimens thereon.

4. The specimen zone mat of claim 2, wherein the one or more grid lines divide the first surface into six to eight designated sections for placing one or more specimens thereon.

5. The specimen zone mat of claim 1, wherein the body portion is non-flammable.

6. The specimen zone mat of claim 1, wherein the writable material includes at least a vinyl material.

7. The specimen zone mat of claim 1. wherein the writeable material includes at least a polyester.

8. The specimen zone mat of claim 1, wherein the non-slip material includes at least a rubber material.

9. The specimen zone mat of claim 1, wherein the first and second surfaces are made of a mixture of both writable and non-slip materials.

10. The specimen zone mat of claim 1, wherein the first surface includes a label of “Specimen Zone” in a center portion thereof.

11. The specimen zone mat of claim 1, wherein the body portion is foldable in half.

12. The specimen zone mat of claim 1, wherein the body portion is colored in a bright color including light purple or lime green.

13. The specimen zone mat of claim 12, wherein the body portion is colored in a neon color.

14. The specimen zone mat of claim 1, wherein the body portion has an approximate size of no more than about 1 meter in length, no more than about 1 meter in width and no more than about 10 cm in thickness.

15. The specimen zone mat of claim 1. wherein the body portion is impermeable to water based fluids.

16. The specimen zone mat of claim 1, wherein the specimen zone mat is in a sterile, single use package
